(19) United States
(12) Patent Application Publication
(10) Pub. No.: US 2023/0210416 A1
(43) Pub. Date: Jul. 6, 2023

(54) SYSTEM AND METHOD OF TOUCH SENSING USING PHYSIOLOGICAL OR BIOCHEMICAL SENSORS

(71) Applicant: JOYSON SAFETY SYSTEMS ACQUISITION LLC, Aubum Hills, MI (US)

(72) Inventors: Salvatore BRAUER, Bloomfield, MI (US); Leonard CECH, Brighton, MI (US)

(21) Appl. No.: 18/009,512

(22) PCT Filed: Jun. 9, 2021
(86) PCT No.: PCT/US2021/036588

§ 371 Date: Dec. 9, 2022

Related U.S. Application Data
(60) Provisional application No. 63/036,682, filed on Jun. 9, 2020.

(51) Int. Cl. A61B 5/1455; A61B 5/00; A61B 5/1495

(52) U.S. Cl. CPC A61B 5/1455; A61B 5/7455; A61B 5/1495; A61B 2090/065

(57) ABSTRACT

Disclosed herein are systems, assemblies and methods of a sensor system that applies a correction factor to spectra information based on a force associated with a touch from a person applying the touch to the sensor system.

### CROSS REFERENCE TO RELATED APPLICATION

This application claims priority to and benefit of U.S. provisional patent application No. 63/036,682 filed Jun. 9, 2020, which is fully incorporated by reference and made a part hereof.

### BACKGROUND

Touch based, non-invasive physiological and biochemical sensor technology is advancing quickly including smaller size, improved human-machine interface (HMI) interaction, higher signal-to-noise ratio (SNR), reduced measurement times and new information extraction techniques. This includes the inclusion of such sensors on cell phones and/or watches, re-usable medical devices (e.g. pulse oximeters), and the like. As an example, to optimize the measurement quality for non-invasive finger touch sensors, various methods, including capacitive sensors to localize the placement of the finger in a proper configuration and provide haptic, audible feedback, have been disclosed. As a second example, pulse oximeter systems attempt to normalize the applied force of the finger on the sensor probe by using a spring-loaded finger probe clip. Once placed on the finger, the clip applies a nominal and uniform pressure on the finger/probe interface.

However, the ability, accuracy and precision to extract useful information from such sensors (for example, detecting one or more specific bio-chemical analytes and/or the concentration of those analytes) is a function of numerous variables relating to the test subject, test location on the subject (e.g. palmar side of finger) and the HMI of the test (sensor probe size, application force, sensor feedback during the measurement, etc.). For conventional systems, such as the examples described above, the applied force can vary based on the size and applied location on the finger, the spring mechanical characteristics, and the age and loading history of the spring. Such variations can vastly reduce the ability to detect one or more physiological properties or one or more biochemical analytes and/or the accuracy and precision of measuring a physiological property and/or analyte(s). For example, the contact pressure can vary the absolute error of a blood oxygen measure (SpO2) by up to 10% or more This is significant, particularly as the measure is derived from a ratio of several difference wavelength reflectivity measures over time.

Therefore, systems and methods are desired that overcome challenges in the conventional art, some of which are described herein. For example, systems and methods of touch sensing that can be used in a switch are desired. More specifically, systems and methods of physiological and/or biochemical non-invasive touch sensing for inclusion in a switch are desired. It may also be desirable for the switch to include an active haptic feedback system (e.g. noise/vibration) which can be activated as a means to inform the person pushing the button of a change in state.

### SUMMARY

Disclosed herein are systems, assemblies and methods of a sensor assembly that determines a force level and spectra information of a touch and applies a spectra correction factor to the spectra information based on the force level.

Additional advantages will be set forth in part in the description which follows or may be learned by practice. The advantages will be realized and attained by means of the elements and combinations particularly pointed out in the appended claims. It is to be understood that both the foregoing general description and the following detailed description are exemplary and explanatory only and are not restrictive, as claimed.

### DRAWINGS

The accompanying drawings, which are incorporated in and constitute a part of this specification, illustrate embodiments and together with the description, serve to explain the principles of the methods and systems:

FIG. 1A is an illustration of a block diagram of an exemplary spectra sensor system;

FIG. 1B is a chart of exemplary chemical bonds related to absorbance spectra in a portion of the infrared electromagnetic region;

FIG. 1C is an illustration of a block diagram of an exemplary spectra sensor system further comprising one or more haptic exciters;

FIG. 1D is an illustration of a block diagram of an exemplary spectra sensor system further comprising one or more haptic exciters, one or more force sensors, and a touch plate;

FIG. 2A illustrates an exemplary switch assembly;

FIG. 2B illustrates an exemplary switch assembly that further comprises a touch plate and a haptic exciter;

FIG. 3 is a flowchart of a method of applying a correction factor to spectra information based on a force level of a touch applied to the spectra sensor;

FIG. 4 illustrates an exemplary computer that can be used for executing software for determining a touch force applied to the spectra sensor and spectra information from the spectra sensor about the person applying the touch;

FIG. 5A is an image of an exemplary sensor spectra measurement system comprising a spectrometer sitting on microscale to measure force in grams synchronously in time with spectra measurements.

FIG. 5B is a graph showing absorbance spectra created by the spectrometer of FIG. 5A. The spectrometer was placed in the middle of the scale and zeroed out (e.g. a baseline spectra established with no finger applied force). A finger was then placed on the device with applied force to obtain: 50 g, 100 g, 120 g, 150 g, 200 g, 250 g, 300 g, 350 g, 400 g, 450 g, and 500 g respectively. The values and spectra were then put into a chemometrics modeling software to find correlation of finger surface spectra changes to applied force changes;

FIG. 6 is a graph showing predicted (force in grams, y-axis) versus measured (force in grams, x-axis) correlation using only the second latent variable from a principal component analysis with three latent variables using the spectra from FIG. 5B;

FIG. 7 is a graph showing the RMSEC (residual mean square error of calibration error) and RMSECV (residual mean square error of calibration error variance) for the prediction model for force and spectra correlation as a function of # of latent variables used to derive the predication model. It shows that these errors approach zero. Verifying that the relation between applied force and finger spectra can be correlated and a prediction model can be created for the applied force of the index finger for one subject;

FIG. 8 is a graph showing spectrometer data (X=wavelength (in nm), Y=reflectivity). It shows 100 spectra of 4 different people at 10 different force levels according to Table 1. Total of 400 spectra in the dataset;

FIG. 9 shows a derived PLS model prediction, where the x axis shows the applied force and the y axis shows the derived models (shapes correspond to the applied force, circles are lowest, stars are highest). In order to estimate the validity of the model another method is used called partial least squares discriminant analysis (PLSDA);

FIG. 10 shows Receiver Operating Characteristic (ROC) plots showing correlation between force and spectra grouped into three distinct categories based off Table 1; and

FIG. 11 shows the classification result of the prediction model using a truth table format. It demonstrates the ability to “predict” force based on a previously defined chemometrics model. It also shows the results using cross validation. The vast majority of spectra are selected in the correct force range based on their spectra.

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes all values from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combination and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The present methods and systems may be understood more readily by reference to the following detailed description of preferred embodiments and the Examples included therein and to the Figures and their previous and following description.

It is known that pressure on living tissue forces spectra information of the living tissue to change. Such spectra information may include, for example, physiological and/or biochemical spectra information including, but not limited to, blood chemistry (e.g. glucose level, blood ethanol, hydration level, . . . ), physiological measures (e.g. blood pressure, blood oxygen levels and patterns, . . . ), thickness of the skin, compressed capillaries, and reduced blood flow to sensor contact area. Taking this concept into consideration, using a sensor system that can capture physiological and/or biochemical spectra information about a touch applied to a spectra sensor and that can capture a force level of the touch applied to the spectra sensor, it is possible to model the changes in spectra. Using a mathematical chemometric model such as Partial Least Squares (PLS), for example, a correlation can be obtained between force and the spectra taken by the spectra sensor. This allows for effectively eliminating the force variable for testing by using compensation methods if variations in force cause measurement errors in physiological or biochemical measurements. Or, this can be used to display force applied on the spectra sensor so that living tissue subjects can reach and/or maintain a nominal force for physiological or biochemical measurements to be made accurately. Measurements can be omitted when the force does not meet a determined high and/or low threshold or does not fall within a specified range.

Correction factors can be derived and applied during real time measurements by measuring force data for all touch-based measurements and deriving a detection or measurement model. A switch can be provided that allows a method to measure force with no movement of the touch interface. Such a switch can be used, for example, with an optical measurement path for any near infrared spectrometer. This avoids optical path errors due to movement.

Furthermore, such a switch also may include haptic excitation at the sensor system interface. Applying a vibration (random or otherwise) at a determined average amplitude and frequency can be used to alert the person applying the touch that the required force level has or has not been reached or to indicate the touch is within or not within the required force range for an effective spectra measurement.

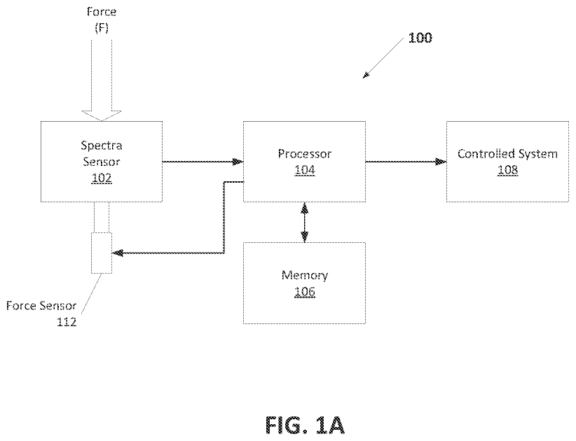

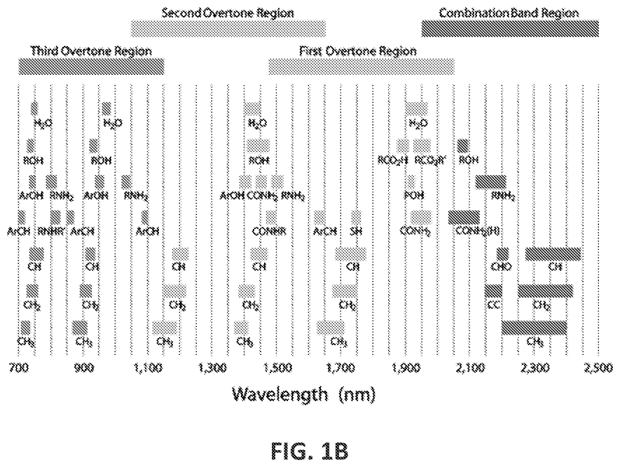

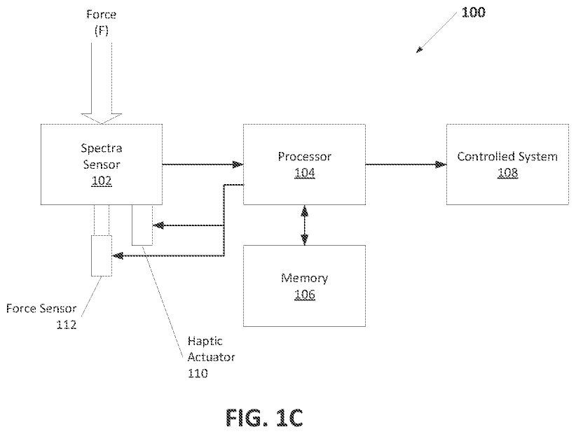

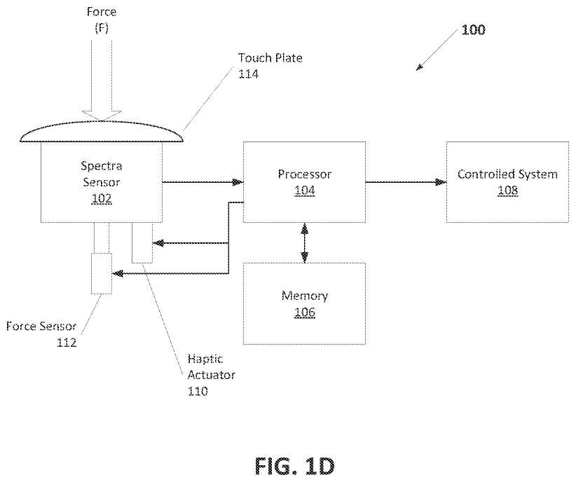

FIG. 1A is an illustration of a block diagram of an exemplary spectra sensor system 100. Such a system 100 may comprise a switch. In some instances, such a switch may be used in a vehicle to control vehicle systems (e.g., turn on/off or adjust entertainment systems, environmental controls, windshield wipers, lights, seating adjustments, engine on/off, and the like). In some instances, such a switch may be embedded in a driving apparatus (e.g., a steering wheel) of a vehicle.

As shown in FIG. 1A, one embodiment of the spectra sensor system 100 comprises a spectra sensor 102. The spectra sensor 102 captures physiological and/or biochemical spectra information about a touch applied to the spectra sensor 102. In some instances, the sensor captures physiological and/or biochemical spectra information about the touch applied to the sensor using spectroscopy. For example, the spectra sensor 102 may capture physiological and/or biochemical spectra information about a touch applied to the spectra sensor 102 using one or more of visible light spectroscopy, infrared spectroscopy, near-infrared spectroscopy, mid infrared spectroscopy, fluorescence spectroscopy, nuclear magnetic resonance spectroscopy, and the like. In some instances, the spectra sensor 102 comprises a visible light and near-infrared spectrometer with a range of 300-2600 nm or more. For example, the near-infrared spectrometer may comprise a Michelson, Grating Filter, diode array, organic silicon, or a Fabry-Perot near-infrared spectrometer.

The spectra sensor system 100 of FIG. 1A further comprises a memory 106 in communication with a processor 104. The processor 104 is communicatively coupled with the memory 106, one or more force sensors 112, and the spectra sensor 102. The processor 104 executes computer-readable instructions stored on the memory 106. In one instance, the computer-readable instructions cause the processor to receive the spectra information from the spectra sensor 102 and cause an action to occur based on the spectra information by the processor 104 sending a control signal to a controlled system 108. The one or more force sensors 112 measure force (i.e., touch force) applied to either the spectra sensor 102 or a touch plate (described herein). In some implementations, the one or more force sensors 112 may comprise micro electro-mechanical sensors (MEMS) that provide an output signal that corresponds with an amount of force received by the sensors. For example, the MEMS force sensors are able to detect force with as little as 2 microns of displacement. In other instances, the one or more force sensors 112 may comprise capacitive sensors, piezoresistive sensors, mechanical sensors, or other suitable sensors for identifying a touch event and measuring an associated force.

The one or more force sensors 112 may be used to develop a calibration model. A correction factor is derived from the calibration model and the correction factor is applied to the physiological and/or biochemical spectra information captured by the spectra sensor 102 about the touch applied to the sensor 102 as a function of time based on changing force. In some instances, the calibration model may be derived for an individual subject and/or for a statistical cross-sample of a wide range of subjects.

In some instances, the spectra information received from the spectra sensor 102 is processed by the processor 104 using chemometric analysis. In some instances, this may comprise using a predictive model that has been trained to correlate spectral features with force values for a dataset of touch forces. In various instances, the chemometric analysis may comprise one or more of partial least squares, principal component analysis, principal component regression, and multi-linear regression.

In some instances, the spectra sensor system 100 is analyzing, using the chemometric analysis, constituents/anomalies/changes in a subject's blood stream (the subject being a person applying a touch force to the spectra sensor 102 or the optional touch plate 114, described below). Specifically, the spectra sensor system 100 may be measuring differing bonds of molecules. For example, it may be measuring organic chemical bonds such as, but not limited to, C—H, N—H, and O—H bonds. A change in the amount of bonds in the blood of the subject causes the absorbance of the spectra to change, which can be measured using the chemometric analysis. This change can be used to determine if there are unwanted components in the subject's physiology. FIG. 1B is a chart of bonds related to absorbance spectra. If there are changes in the amount of these bonds, it can be detected in the spectral measurements using the chemometric analysis (e.g. when people have high blood sugar C₆H₁₂O₆ they will have different, predictable measurements vs people who don't). Another example is alcohol concentration C₂H₅OH. For example, if the spectra sensor 102 determines using spectra information of a person touching or pushing the spectra sensor 102 that the person's alcohol content is too high, the system may cause an action to be taken, or not taken by the controlled system 108. For example, if the alcohol content is determined to exceed a certain threshold, the spectra sensor system 100 won't allow an ignition system of an automobile (e.g., the controlled system 108) to start.

Referring now to FIG. 1C, in some instances the spectra sensor system 100 may further comprise a haptic exciter or actuator 110 in communication with the processor 104. The processor 104 causes the haptic exciter 110 to generate a haptic feedback that can be felt at the touch point of the spectra sensor 102 or a touch plate in response to a determined touch force. In some instances, the haptic feedback is generated by the processor 104 in response to the determined touch force exceeding, meeting, or not meeting a defined threshold or range. In some instances, the haptic feedback is generated only after the determined touch force exceeds the threshold for a predetermined period of time. In some instances, the haptic feedback is generated continuously while the force exceeds or meets a defined threshold or range, indicating to the user that the spectra sensor 102 is continuously reading spectra information and therefore the user should continue to apply the force.

Referring now to FIG. 1D, in some instances, as noted herein, the spectra sensor system 100 may further optionally comprise a touch plate 114 in communication with the spectra sensor 102. When provided, the touch is applied to the touch plate 114. In some instances, neither the touch plate 114 nor the spectra sensor 102 move when the touch forces are applied and, in other instances, the touch plate 114 and spectra sensor 102 are displaced by only microns when the touch forces are applied and move, for example, less than 10 microns. In some instances, the touch plate is configured and/or comprised of suitable materials to allow the spectra sensor 102 to monitor a change in an analyte of the person applying the touch to the touch plate 114. Such materials include, but are not limited to, polycarbonate, which is transparent in the spectral region of interest (e.g. infrared).

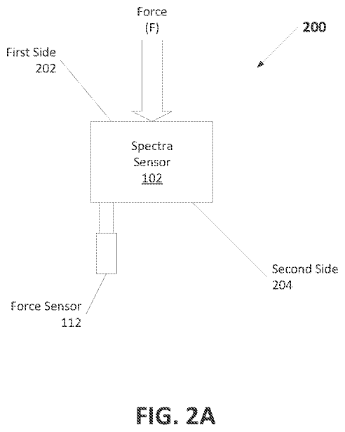

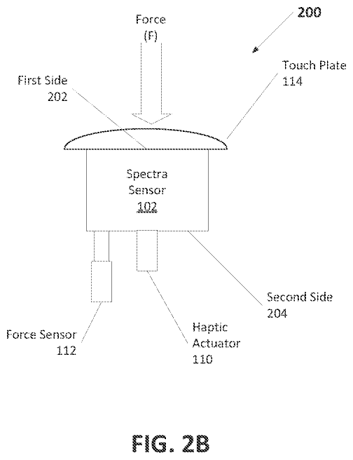

FIG. 2A illustrates an exemplary switch assembly 200. One embodiment of the switch assembly 200 may comprise a spectra sensor 102 having a first side 202 and a second side 204. As described herein, the sensor 102 captures physiological and/or biochemical spectra information about a touch applied to the sensor 102 by monitoring a change in an analyte of a person applying the touch. The switch assembly also includes one or more force sensors 112. The one or more force sensors 112 are used to determine a force level of a touch, and the spectra sensor 102 obtains spectra information of the touch. Collectively, the force level and the spectra information are used to apply a spectra correction factor to the spectra information based on the force level. The correction factor is applied to the physiological and/or biochemical spectra information captured by the sensor 102 about the touch applied to the sensor as a function of time based on changing force.

In the embodiment of a switch assembly 200 shown in FIG. 2B, the switch assembly 200 further comprises an optional haptic actuator 110 in communication with at least a portion of the spectra sensor 102. The haptic actuator 110 causes a haptic feedback to at least the first side 202 of the spectra sensor 102 subsequent to the touch being applied to the first side 202. The embodiment of FIG. 2B further comprises a touch plate 114. As noted herein, the touch plate 114 is comprised of suitable materials to allow the spectra sensor 102 to monitor a change in an analyte of the person applying the touch to the touch plate 114. As a non-limiting example, the touch plate may comprise all or a portion of an automobile start/stop button that would not look any different than conventional start/stop buttons to the user, but would allow the system to properly analyze the spectra information from the user. For instance, if alcohol content is too high, the system won't allow the car to start.

Generally, the switch assembly 200 is in communication with a processor 104 having a memory 106. The processor 104 and/or memory 106 may be a part of the switch assembly 200 or may be separate from the switch assembly 200. The processor 104 executes the computer-readable instructions stored on the memory 106 that cause the processor 104 to receive the spectra information from the spectra sensor 102, where the spectra information is associated with the change in the analyte of the person applying the touch. Determining the change in the analyte of the person applying the touch comprises determining constituents/anomalies/changes in the subject's blood stream. Determining constituents/anomalies/changes in the subject's blood stream comprises measuring differing bonds of molecules. For example, organic chemical bonds, including C—H, N—H, and O—H bonds, may be measured. A change in the amount of bonds in the blood creates a change in spectra information. In some instances, the change in spectra information indicates unwanted components in a subject's physiology (e.g., alcohol, drugs, etc.).

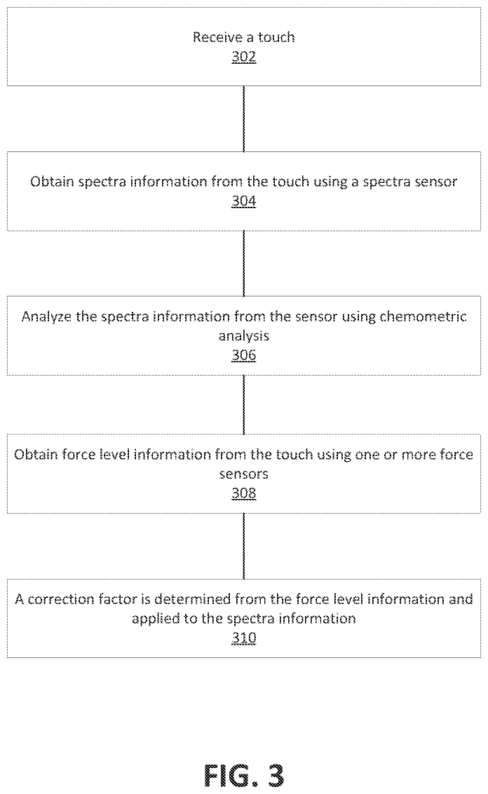

The disclosed systems and assemblies enable a method of determining spectra information from a person applying a touch using chemometric analysis and applying a correction factor to the spectra information based on the force level of the touch. In one aspect, as shown in the flowchart of FIG. 3, this is done at step 302, receiving a touch. This touch may be to a spectra sensor, or to a touch plate, as described herein. At step 304, spectra information from the touch is obtained by the spectra sensor. At step 306, the spectra information is provided to a processor, where the processor analyzes the spectra information using chemometric analysis. At step 308, force level information about the touch is obtained using one or more force sensors. At step 310, a correction factor is determined and applied to the spectra information based on the force level.

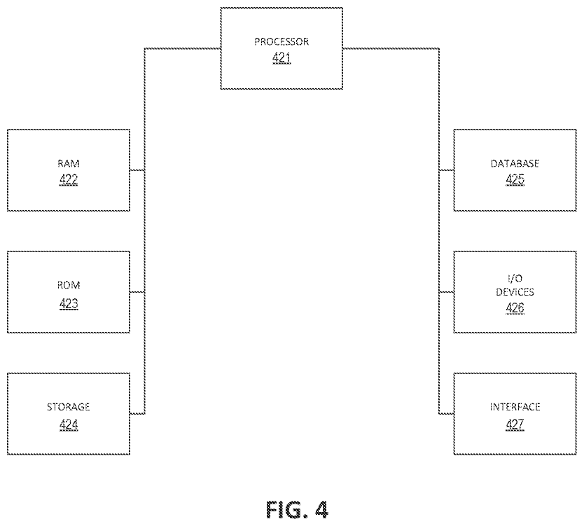

The systems and assemblies described herein have been described above as comprised of units. One skilled in the art will appreciate that this is a functional description and that the respective functions can be performed by software, hardware, or a combination of software and hardware. A unit can be software, hardware, or a combination of software and hardware. The units can comprise software for analysis of spectra information received from a spectra sensor. In one exemplary aspect, the units can comprise an analyzer and/or a data analysis computer that comprises one or more computing devices that each comprise a processor 421 as illustrated in FIG. 4 and described below. As used herein, processor refers to a physical hardware device that executes encoded instructions for performing functions on inputs and creating outputs.

FIG. 4 illustrates an exemplary computer that can be used for executing software for applying a correction factor to spectra information based on a detected force level of a touch applied to a spectra sensor by a user. As used herein, “computer” may include a plurality of computers. The computers may include one or more hardware components such as, for example, a processor 421, a random access memory (RAM) module 422, a read-only memory (ROM) module 423, storage 424, a database 425, one or more input/output (I/O) devices 426, and an interface 427. Alternatively, and/or additionally, the computer may include one or more software components such as, for example, a computer-readable medium including computer executable instructions for performing a method associated with the exemplary embodiments. It is contemplated that one or more of the hardware components listed above may be implemented using software. For example, storage 424 may include a software partition associated with one or more other hardware components. It is understood that the components listed above are exemplary only and not intended to be limiting.

Processor 421 may include one or more processors, each configured to execute instructions and process data to perform one or more functions associated with a computer for executing software to perform a method of determining and applying a correction factor to spectra information based on the determined touch force applied to the spectra sensor by a user. Processor 421 may be communicatively coupled to RAM 422, ROM 423, storage 424, database 425, I/O devices 426, and interface 427. Processor 421 may be configured to execute sequences of computer program instructions to perform various processes. The computer program instructions may be loaded into RAM 422 for execution by processor 421.

RAM 422 and ROM 423 may each include one or more devices for storing information associated with the operation of processor 421. For example, ROM 423 may include a memory device configured to access and store information associated with the computer, including information for identifying, initializing, and monitoring the operation of one or more components and subsystems. RAM 422 may include a memory device for storing data associated with one or more operations of the processor 421. For example, ROM 423 may load instructions into RAM 422 for execution by processor 421.

Storage 424 may include any type of mass storage device configured to store information that processor 421 may need to perform processes consistent with the disclosed embodiments. For example, storage 424 may include one or more magnetic and/or optical disk devices, such as hard drives, CD-ROMs, DVD-ROMs, or any other type of mass media device.

Database 425 may include one or more software and/or hardware components that cooperate to store, organize, sort, filter, and/or arrange data used by the computer and/or processor 421. For example, database 425 may store data related to the analysis software. The database may also contain data and instructions associated with computer-executable instructions for performing methods of determining and applying a correction factor to spectra information based on the determined touch force applied to the spectra sensor by a user. It is contemplated that database 425 may store additional and/or different information than that listed above.

I/O devices 426 may include one or more components configured to communicate information with a user associated with a computer. For example, I/O devices may include a console with an integrated keyboard and mouse to allow a user to maintain a database of analyte change/applied force correlations specific to a subject or a set of subjects, and the like. I/O devices 426 may also include a display including a graphical user interface (GUI) for outputting information on a monitor. I/O devices 426 may also include peripheral devices such as, for example, a printer, a user-accessible disk drive (e.g., a USB port, a floppy, CD-ROM, or DVD-ROM drive, etc.) to allow a user to input data stored on a portable media device, a microphone, a speaker system, or any other suitable type of interface device.

Interface 427 may include one or more components configured to transmit and receive data via a communication network, such as the Internet, a local area network, a workstation peer-to-peer network, a direct link network, a wireless network, or any other suitable communication platform. For example, interface 427 may include one or more modulators, demodulators, multiplexers, demultiplexers, network communication devices, wireless devices, antennas, modems, and any other type of device configured to enable data communication via a communication network.

The figures illustrate the architecture, functionality, and operation of possible implementations of systems, methods and computer program products according to various implementations of the present invention. In this regard, each block of a flowchart or block diagram may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustrations, and combinations of blocks in the block diagrams and/or flowchart illustrations, can be implemented by special purpose hardware-based systems that perform the specified functions or acts, or combinations of special purpose hardware and computer instructions.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The implementation was chosen and described in order to best explain the principles of the invention and the practical application, and to enable others of ordinary skill in the art to understand the invention for various implementations with various modifications as are suited to the particular use contemplated.

Any combination of one or more computer readable medium(s) may be used to implement the systems and methods described herein above. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the computing device of a system's computer, partly on the system's computer, as a stand-alone software package, partly on the system's computer and partly on a remote computer or entirely on the remote computer or server. In the latter scenario, the remote computer may be connected to the system's computer through any type of network, including a local area network (LAN) or a wide area network (WAN), or the connection may be made to an external computer (for example, through the Internet using an Internet Service Provider). A non-limiting example of a system's computer includes a computer or computing device in a vehicle, In such an instance, the system's computer may be one that is dedicated to the embodiments described herein, or it may be a computer or processing device used to communicate with, monitor, and/or control various vehicle systems.

Aspects of the present invention are described herein with reference to flowchart illustrations and/or block diagrams of methods, apparatus (systems) and computer program products according to implementations of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

### Examples

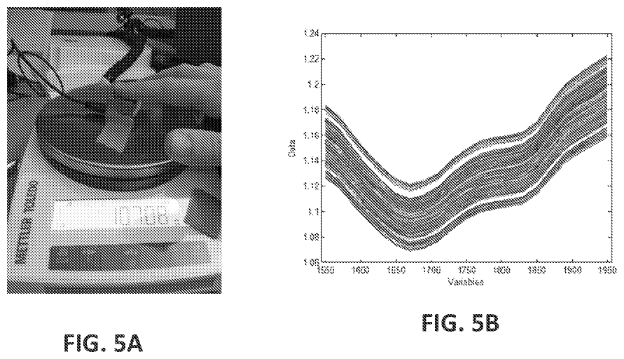

FIG. 5A is an image of an exemplary sensor system comprising a spectrometer (i.e. spectra sensor) sitting on a microscale (i.e. force sensor) to measure force in grams.

FIG. 5B is a graph showing absorbance spectra created by the spectrometer from FIG. 5A. The spectrometer was placed in the middle of the scale and zeroed out. A finger was then placed on the device with applied pressure to obtain: 50 g, 100 g, 120 g, 150 g, 200 g, 250 g, 300 g, 350 g, 400 g, 450 g, and 500 g respectively. The values and spectra were then put into a PLS modeling software to find correlation.

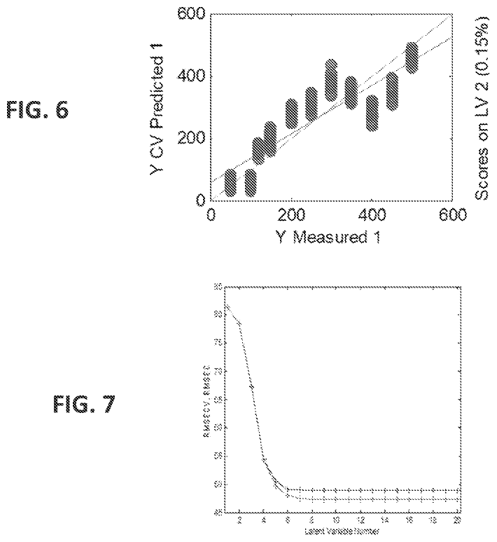

FIG. 6 is a graph showing predicted versus measured correlation with three latent variables on one subject. FIG. 7 is a graph showing that RMSEC and RMSECV for force and spectra can be correlated and a prediction model can be created for one subject.

FIG. 5A is an image of an exemplary sensor spectra measurement system comprising a spectrometer sitting on microscale to measure force in grams synchronously in time with spectra measurements.

FIG. 5B is a graph showing absorbance spectra created by the spectrometer of FIG. 5A. The spectrometer was placed in the middle of the scale and zeroed out (e.g., a baseline spectra established with no finger applied force). A finger was then placed on the device with applied force to obtain: 50 g, 100 g, 120 g, 150 g, 200 g, 250 g, 300 g, 350 g, 400 g, 450 g, and 500 g respectively. The values and spectra were then put into a chemometrics modeling software to find correlation of finger surface spectra changes to applied force changes.

FIG. 6 is a graph showing predicted (force in grams, y-axis) versus measured (force in grams, x-axis) correlation using only the second latent variable from a principal component analysis with three latent variables using the spectra from FIG. 5B.

FIG. 7 is a graph showing the RMSEC (residual mean square error of calibration error) and RMSECV (residual mean square error of calibration error variance) for the prediction model for force and spectra correlation as a function of # of latent variables used to derive the predication model. It shows that these errors approach zero. Verifying that the relation between applied force and finger spectra can be correlated and a prediction model can be created for the applied force of the index finger for one subject.

A second experiment using 4 people instead of 1 was carried out to verify that a calibration model could be derived over a variation of subjects applying measured force on the spectrometer and collecting spectral sample sets for fixed applied force.

Table 1: Force Sensor Output to Newtons and Grams

TABLE 1
Force Sensor Output to Newtons and Grams

| Force (N) | Mass (g) |
|---|---|
| 0.15 | 12 |
| 0.31 | 27 |
| 0.51 | 47 |
| 0.80 | 73.02 |
| 1.21 | 108 |
| 1.79 | 162 |
| 2.68 | 244.13 |
| 4.53 | 412.3 |
| 9.13 | 830.84 |
| 13.38 | 1217.63 |

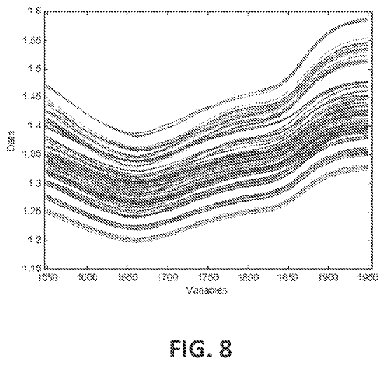

FIG. 8 is a graph showing spectrometer data (X=wavelength (in nm), Y=reflectivity). It shows 100 spectra of 4 different people at 10 different force levels according to Table 1. Total of 400 spectra in the dataset.

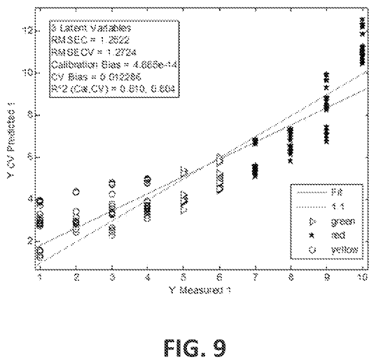

FIG. 9 shows a derived PLS model prediction, where the x axis shows the applied force and the y axis shows the derived models (shapes correspond to the applied force, circles are lowest, stars are highest). In order to estimate the validity of the model another method is used called partial least squares discriminant analysis (PLSDA).

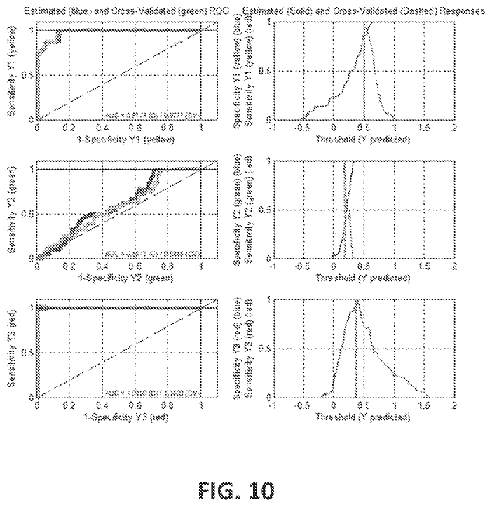

FIG. 10 shows Receiver Operating Characteristic (ROC) plots showing correlation between force and spectra grouped into three distinct categories based off Table 1.

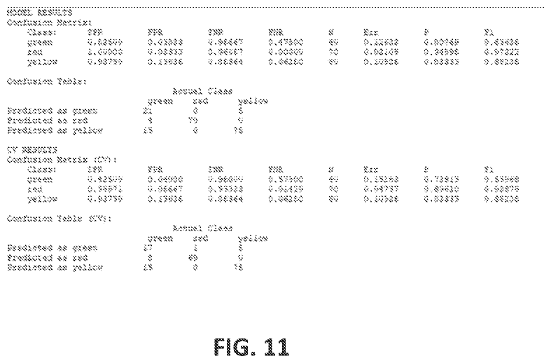

FIG. 11 shows the classification result of the prediction model using a truth table format. It demonstrates the ability to “predict” force based on a previously defined chemometrics model. It also shows the results using cross validation. The vast majority of spectra are selected in the correct force range based on their spectra. A confusion matrix creates a table of results showing True Positive, False Positive, True Negative, and False Negative rates (TPR, FPR, TNR, and FNR) as a matrix for each class modeled in an input model, where:
TPR: proportion of positive cases that were correctly identified (Sensitivity), =P/(TP+FN)
FPR: proportion of negatives cases that were incorrectly classified as positive, =P/(FP+TN)
TNR: proportion of negatives cases that were classified correctly (Specificity), =TN/(TN+FP)
FNR: proportion of positive cases that were incorrectly classified as negative, =N/(FN+TP)

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

While the methods and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

Throughout this application, various publications may be referenced. The disclosures of these publications in their entireties are hereby incorporated by reference into this application in order to more fully describe the state of the art to which the methods and systems pertain. For example, the embodiments disclosed herein may be used in switch assemblies such as those described in U.S. Patent Publication 2018/0188876 published Jul. 5, 2018; and U.S. Patent Publication 2018/0190449 published Jul. 5, 2018; which are each fully incorporated by reference and also attached hereto as appendices and made a part hereof.

It will be apparent to those skilled in the art that various modifications and variations can be made without departing from the scope or spirit of the appended claims. Other embodiments will be apparent to those skilled in the art from consideration of the specification and practice disclosed herein. It is intended that the specification and examples be considered as exemplary only, with a true scope and spirit being indicated by the following claims.

1. An analyte detection system comprising:
a spectra sensor, wherein the spectra sensor captures physiological and/or biochemical spectra information about a touch applied to the spectra sensor;
a force sensor, wherein the force sensor determines a force level of the touch applied to the spectra sensor;
a memory, wherein the memory stores computer-readable instructions; and
a processor communicatively coupled with the memory, the spectra sensor, and the force sensor, wherein the processor executes the computer-readable instructions stored on the memory, the computer-readable instructions causing the processor to:
receive the spectra information from the spectra sensor;
receive the force level from the force sensor;
determine if the force level is within a threshold force range and, if the force level is within the threshold force range, apply one or more spectra correction factors to the spectra information based on the force level.

2. (canceled)

3. (canceled)

4. The analyte detection system of claim 1, wherein applying the one or more spectra correction factors to the spectra information based on the force level comprises determining the one or more spectra correction factors using chemometric analysis.

5. (canceled)

6. (canceled)

7. The analyte detection system of claim 4, wherein the chemometric analysis detects a change in an analyte of a person applying the touch.

8. The analyte detection system of claim 7, wherein detecting the change in the analyte comprises determining constituents/anomalies/changes in the person's blood stream.

9. (canceled)

10. (canceled)

11. (canceled)

12. (canceled)

13. The analyte detection system of claim 1, wherein the spectra sensor captures physiological and/or biochemical spectra information about the touch applied to the spectra sensor using spectroscopy.

14. The analyte detection system of claim 13, wherein the spectroscopy comprises one or more of infrared spectroscopy, near-infrared spectroscopy, mid-infrared spectroscopy, and nuclear magnetic resonance spectroscopy.

15. (canceled)

16. (canceled)

17. The analyte detection system of claim 1, further comprising a touch plate in communication with the spectra sensor, wherein the touch is applied to the touch plate and touch force levels on the touch plate are determined by the force sensor.

18. (canceled)

19. The analyte detection system of claim 1, further comprising a haptic actuator in communication with the processor, wherein the haptic actuator generates a haptic feedback in response to the determined touch force level.

20. (canceled)

21. (canceled)

22. (canceled)

23. (canceled)

24. The analyte detection system of claim 1, further comprising a calibration model, wherein the one or more spectra correction factors are derived from the calibration model and the one or more spectra correction factors are applied to the physiological and/or biochemical spectra information as a function of time based on changing force.

25. (canceled)

26. A switch assembly comprising:
a spectra sensor, wherein the spectra sensor has a first side and a second side, wherein the spectra sensor captures physiological and/or biochemical spectra information about a touch applied to the spectra sensor first side by monitoring a change in an analyte of a person applying the touch; and
one or more force sensors, wherein the one or more force sensors determine a force level of the touch applied to the spectra sensor first side,
wherein if the force level is within a threshold force range, one or more spectra correction factors is applied to the spectra information based on the force level.

27. The switch assembly of claim 26, wherein the switch assembly is in communication with a processor having a memory, wherein the processor executes the computer-readable instructions stored on the memory, the computer-readable instructions causing the processor to:
receive the spectra information from the spectra sensor, said spectra information associated with the change in the analyte of the person applying the touch;
receive the force level from the force sensor;
determine if the force level is within the threshold force range and, if the force level is within the threshold force range, apply the one or more spectra correction factors to the spectra information based on the force level; and
take an action based on the spectra information.

28. (canceled)

29. The switch assembly of claim 26, wherein applying the one or more spectra correction factors to the spectra information based on the force level comprises determining the one or more spectra correction factors using chemometric analysis.

30. (canceled)

31. (canceled)

32. The switch assembly of claim 29, wherein the chemometric analysis detects a change in an analyte of a person applying the touch.

33. The switch assembly of claim 32, wherein detecting the change in the analyte comprises determining constituents/anomalies/changes in the person's blood stream.

34. (canceled)

35. (canceled)

36. (canceled)

37. (canceled)

38. The switch assembly of claim 26, wherein the spectra sensor captures physiological and/or biochemical spectra information about the touch applied to the spectra sensor using spectroscopy.

39. The switch assembly of claim 38, wherein the spectroscopy comprises one or more of infrared spectroscopy, near-infrared spectroscopy, mid-infrared spectroscopy, and nuclear magnetic resonance spectroscopy.

40. The switch assembly of claim 26, wherein the spectra sensor comprises a visible and infrared spectrometer with a range of 300-2600 nm.

41. (canceled)

42. The switch assembly of claim 26, further comprising a touch plate in communication with the spectra sensor, wherein the touch is applied to the touch plate and touch force levels on the touch plate are determined by the force sensor.

43. (canceled)

44. The switch assembly of claim 26, further comprising a haptic actuator in communication with the processor, wherein the haptic actuator generates a haptic feedback in response to the determined touch force level.

45. (canceled)

46. (canceled)

47. (canceled)

48. (canceled)

49. A method of determining applied force to a sensor using the systems and assemblies described in claim 1, said method comprising:
receiving a touch applied to a sensor;
receiving spectra information from the received touch, said spectra information associated with the person applying the touch;
analyze the spectra information from the sensor using chemometric analysis;
obtain force level information from the touch using one or more force sensors;
determine one or more correction factors from the force level information; and
apply the one or more correction factors to the spectra information.
